(19) United States
(12) Patent Application Publication
(10) Pub. No.: US 2022/0331247 A1
(43) Pub. Date: Oct. 20, 2022

(54) Polymeric micelle, methods of production and uses thereof

(71) Applicant: Association for the Advancement of Tissue Engineering and Cell Based Technologies & Therapies A4TEC, Barco Gmr (PT)

(72) Inventors: Ana Cláudia FERNANDES LIMA, Barco Guimarães (PT); Rui Luís GONÇALVES DOS REIS, Barco Guimarães (PT); Helena Susana DA COSTA MACHADO FERREIRA, Barco Guimarães (PT); Nuno João MELEIRO ALVES DAS NEVES, Barco Guimarães (PT)

(21) Appl. No.: 17/762,175

(22) PCT Filed: Sep. 22, 2020
(86) PCT No.: PCT/IB2020/058851

§ 371 Date: Mar. 21, 2022

(30) Foreign Application Priority Data
Sep. 23, 2019 (PT) 115792
Dec. 31, 2019 (EP) 19220276.0

(51) Int. Cl. A61K 9/107; A61K 31/573

(52) U.S. Cl. CPC A61K 9/1075; A61K 31/573

(57) ABSTRACT

The present disclosure relates to enzymatic and redox responsive polymeric micelle, its method of production as well as its uses. Specifically, use of the enzymatic- and redox-responsive polymeric micelle for drug delivery.

### TECHNICAL FIELD

The present disclosure relates to an enzymatic and redox responsive polymeric micelle, its method of production as well as its uses. Specifically, use of the enzymatic and redox responsive polymeric micelle for drug delivery.

### BACKGROUND

Inflammation is a fundamental, yet complex, process designed to protect the human body. However, inflammation is also involved in a vast variety of human diseases, such as arthritis, autoimmunity, neurological and cardiovascular diseases, and cancer [1]. Current treatments are based on non-steroidal anti-inflammatory drugs (NSAIDs), corticosteroids and biologic agents. Since they lack specificity and have off-target distribution, currents treatments are also associated with adverse side effects [2].

The therapeutic drawbacks of current treatments open a window of opportunity to design nanomedicines as drug delivery systems, and several formulations have been successfully introduced in the clinic for the treatment of cancer, pain and infectious diseases [1]. Among the clinically translatable nanomedicines, polymeric micelles exhibit several features that favour their utility in drug delivery applications, including its biocompatibility, longevity, high stability in vitro and in vivo, as well as its ability to increase drug bioavailability and accumulation in target tissues [2,3]. Therefore, nanoscale features can be used to enhance cell permeability to allow the nanoparticles to remain in circulation longer and to allow targeted delivery to certain cells. Therefore, the most promising application of nanomaterials is in the targeted, controlled and site-specific drug delivery, which consequently improves drug efficacy, reduces the required dosage amounts and side effects. Nonetheless, there are still some limitations in current nanomedicine treatment methods as accumulation in target tissue is normally less than 10%. This means that drug efficacy is lower as at least part of the drug load is lost during circulation [3,4].

Engineered nanoparticles that respond to pathophysiological conditions such as redox potential and pH have been developed to control the spatiotemporal distribution of the drugs, thus reducing the dosage required and avoid systemic side effects [4,5]. Such conditions can be endogenously present in the body, and intensified or distinctly overexpressed in diseased tissues [6]. Glutathione (GSH) is the key regulator of the intracellular redox state and is required for the detoxification of reactive oxygen species (ROS), peroxides, electrophilic xenobiotic compounds, and for the regeneration of other endogenous and exogenous antioxidants [7,8]. Since concentrations of GSH outside cells is reported to be as low as 2 μM-20 μM whereas GSH levels within cells range from 2 mM to 10 mM, redox-responsive nanomedicines can be used for intracellular drug delivery [9]. In addition, it has been reported that there are higher glutathione reductase (GR) concentrations in inflammatory conditions including cancer and arthritic conditions [10,11].

These facts are disclosed in order to illustrate the technical problem addressed by the present disclosure.

### GENERAL DESCRIPTION

The present disclosure relates to an enzymatic and redox responsive polymeric micelle, its method of production as well as its uses. Specifically, use of the enzymatic and redox responsive polymeric micelle for drug delivery.

An aspect of the present disclosure relates to a drug delivery system comprising enzymatic and redox responsive polymeric micelles (hereinafter “polymeric micelles”) for targeted and controlled drug release. The disclosed polymeric micelles are designed to increase drug therapeutic efficacy while reducing systemic side effects. As such, as compared to current treatment methods, the disclosed polymeric micelles increase drug therapeutic effect and reduce severe side effects by circumventing unnecessary exposure to healthy tissues.

The disclosed polymeric micelles enhance the drug targeted and controlled release. Current treatments are associated with limited efficacy and serious side effects, requiring higher doses and extended periods of treatment in order to obtain a therapeutic effect. The polymeric micelles increase the therapeutic efficacy of the drug and reduce the associated side effects.

Another aspect of the present disclosure relates to the use of the disclosed polymeric micelles to: i) increase the therapeutic index of a hydrophobic drug through encapsulating the drug in the polymeric micelles; ii) reduce systemic side effects through the controlled release profile of the drug and consequently reducing unnecessary exposure to healthy tissues; and iii) increase the therapeutic efficacy of currently used drugs, including anti-inflammatory and many other therapeutic agents.

In an embodiment, polymeric micelles comprising methoxypolyethylene glycol amine-glutathione-palmitic acid (mPEG-GSH-PA) copolymer were produced. The copolymer was synthetized through 2-step reactions. Firstly, methoxypolyethylene glycol amine (mPEG) was covalently linked to glutathione (GSH) using 1-(3-Dimethylaminopropyl)-3-ethylcarbodiimide (EDC) and N-Hydroxysuccinimide (NHS) as coupling agents. Secondly, mPEG-GSH was allowed to react with palmitic acid (PA) in tetrahydrofuran (THF) with 2-(1H-Benzotriazole-1-yl)-1,1,3,3-tetramethylaminium tetrafluoroborate (TBTU) and triethylamine (TEA) acting as catalysers. After copolymer synthesis, polymeric micelles were prepared through nanoprecipitation. Scanning electron microscopy (SEM) and dynamic light scattering (DLS) analyses demonstrated that the polymeric micelles present a spherical shape and a uniform size (polydispersity index—PDI—<0.1) of 100 nm. The polymeric micelles were able to encapsulate a model drug, dexamethasone (Dex), in its hydrophobic compartment. The polymeric micelles have a loading capacity of up to 65%. In physiological conditions, Dex release from the polymeric micelles was low and slow. However, by increasing the concentration of the reducing agent (GSH) to an intracellular concentration level or increasing the activity of Glutathione Reductase (GR) enzyme to values found during inflammation, the Dex release was significantly facilitated (burst release).

In an embodiment, after confirming the responsiveness of the polymeric micelles, biological assays with human endothelial cells, human articular chondrocytes (hACs) and human macrophages were performed to demonstrate the polymeric micelles' cytocompatibility.

In an embodiment, the effect of the polymeric micelles on chondrocytes and macrophages in monoculture and co-culture systems were analysed. Results show that the polymeric micelles promote targeted and sustained release of drugs in the presence of GR and redox media. Polymeric micelles encapsulating Dex exhibit a higher efficacy than free Dex and also reduce the negative effects of the drug in normal cells. As such, polymeric micelles containing Dex can be used in the treatment and/or therapy of inflammatory conditions such as arthritic diseases.

In an embodiment, a linear copolymer comprising methoxypolyethylene glycol amine-glutathione-palmitic acid (mPEG-GSH-PA) was synthetized and the thiol groups of the GSH were oxidized intermolecularly to encapsulate the drug into the micelles. The oxidation of the adjacent GSH in the micelles avoids leakage into surrounding plasma as the disulphide cross-linking provides a barrier against blood dilution. After accumulation in the inflammatory sites via the enhanced permeability and retention (EPR) effect, the drug would undergo quick release triggered by both redox media and GR activity.

In an embodiment, intracellular GSH exchanges with the thiol disulphide bond of glutathione disulfide (GSSG), and the presence of GR promotes disruption of the polymeric micelles to facilitate rapid drug release.

In an embodiment, polymeric micelles function as a carrier for targeted delivery of an anti-inflammatory drug after systemic administration.

In an embodiment, polymeric micelles made of methoxypolyethylene glycol amine-glutathione-palmitic acid (mPEG-GSH-PA) copolymers encapsulate drugs in its hydrophobic compartment.

In an embodiment, the maximum drug entrapment efficacy and in vitro release profile under different stimulus was assessed. Polymeric micelles' cytocompatibility was validated by culturing them in the presence of endothelial and monocytic cell lines, and human articular chondrocytes (hACs). A co-culture model of articular inflammation was established using hACs and stimulated M1 macrophages. The differences in the cell viability and morphology, as well as, the concentration of pro-inflammatory cytokines was evaluated after treatment with polymeric micelles encapsulating Dexamethasone (Dex) or after treatment with the free Dex.

In an embodiment, in an in vitro assay, polymeric micelles are sensitive to a reducing agent (GSH) and an enzyme (GR), since a disruption of the disulphide bonds are observed, releasing the encapsulated drug.

An aspect of the present disclosure relates to polymeric micelle comprising
a palmitic acid to encapsulate a hydrophobic active ingredient forming the micelle core; and
glutathione and polyethylene glycol forming the micelle branch, wherein the polyethylene glycol is bonded to the micelle surface by the glutathione.

In an embodiment, the polymeric micelle comprises a methoxypolyethylene glycol amine-glutathione-palmitic acid copolymer and a hydrophobic active ingredient encapsulated in the micelle core.

In an embodiment, the polymeric micelle is enzymatic and redox responsive.

In an embodiment, the polymeric micelle is for use in medicine.

In an embodiment, the polymeric micelle is a vehicle for drug delivery.

In an embodiment, the polymeric micelle is for use in the treatment or therapy of inflammatory diseases.

In an embodiment, the polymeric micelle is for use in the treatment or therapy of osteoarthritis or rheumatoid arthritis.

In an embodiment, the active ingredient in the polymeric micelle is any hydrophobic drug.

In an embodiment, the hydrophobic drug in the polymeric micelle may be dexamethasone, prednisolone, betamethasone, or combinations thereof.

In an embodiment, the size of the polymeric micelle is determined by scanning electron microscopy (SEM) and dynamic light scattering (DLS) analyses. The size of the micelle is at least 100 nm, preferably from 100 nm to 120 nm, more preferably from 110 nm to 120 nm.

In an embodiment, the polymeric micelle has an encapsulation (or entrapment) efficiency from 30% to 70%, preferably from 61% to 67%.

In an embodiment, the micelle:drug ratio feed weight of the polymeric micelle is from 1:0.2 to 1:0.8 for polymer concentration of 1 mg/mL, preferably 1:0.08 for polymer concentration of 1 mg/mL.

In an embodiment, the amount of hydrophobic active ingredient in the hydrophobic compartment of the polymeric micelle is from 0.2 mg to 6.0 mg, preferably from 4.4 mg to 4.9 mg.

In an embodiment, the polymeric micelle releases the encapsulated (or entrapped) dexamethasone provided that the glutathione reductase concentration is at least 50 mU.

In an embodiment, the polymeric micelle releases the encapsulated dexamethasone provided that the glutathione concentration is at least 20 μM.

Another aspect of the present disclosure relates to a pharmaceutical composition comprising enzymatic and redox responsive polymeric micelles for use in the treatment of inflammatory diseases.

In an embodiment, the concentration of polymeric micelles in the pharmaceutical composition is not more than 50 μg/mL, preferably 50 μg/mL.

In an embodiment, the pharmaceutical composition is a suspension for systemic administration.

Another aspect of the present disclosure relates to the method of producing enzymatic and redox responsive polymeric micelles comprising the following steps:
covalently linking methoxypolyethylene glycol amine and glutathione using coupling agents to form a first copolymer;
adding the first copolymer to palmitic acid in tetrahydrofuran to form a second copolymer;
nano-precipitating the second copolymer to form polymeric micelles.

In an embodiment, the coupling agents used in the of method of producing the polymeric micelles are 1-(3-Dimethylaminopropyl)-3-ethylcarbodiimide and N-Hydroxysuccinimide to form the first copolymer.

In an embodiment, the method of producing the polymeric micelles further comprises:

adding 2-(1H-Benzotriazole-1-yl)-1,1,3,3-tetramethylaminium tetrafluoroborate (TBTU) and triethylamine (TEA) as catalysers when forming the second copolymer.

### BRIEF DESCRIPTION OF THE DRAWINGS

The following figures provide preferred embodiments for illustrating the disclosure and should not be seen as limiting the scope of invention.

FIG. 1A shows the results of FTIR analyses of the methoxypolyethylene glycol (mPEG), Glutathione (GSH), Palmitic acid (PA) and the polymeric micelles (Mic). FIG. 1B shows the size distribution of the polymeric micelles. FIG. 1C shows the results of stability evaluation of polymeric micelles kept in water for 6 months, at 4° C. FIG. 1D shows the SEM micrographs of the polymeric micelles.

FIG. 2 shows the release profile of dexamethasone (Dex) from the polymeric micelles under different artificial milieu: PBS, 10 mM of Glutathione (GSH), and 50 mU of Glutathione Reductase (GR). The analysis was performed at 37° C. and the results captured over a period of 96 hours.

FIG. 3 shows the biological performance of (A) hACs, (B) EA cell line, and (C) THP-1 cell line cultured with different concentrations of micelles: (I) cell viability, (II) cell proliferation and (Ill) total protein synthesis after 1, 3, and 7 days of culture. Asterisk (*) denotes significant differences (p<0.01) compared to the control (0 μg/mL).

FIG. 4 shows the SEM micrographs of the polymeric micelles cultured with (A) hACs, (B) EA cell line and (C) THP-1 cell line in the (I) absence (control) and in the presence of the micelles at different concentrations: (II) 50 and (III) 100 μg/mL. Scale bar 10 μm.

FIG. 5 shows the biochemical performance of (A) hACs and (B) THP-1 cultured in monolayers and hACs co-cultured with activated M1 macrophages after different treatments: control (Ctr, no treatment), micelles encapsulating Dexamethasone (Mic+Dex) and dexamethasone (Dex). The samples were analyzed for (i) cell viability, (ii) cell proliferation, (C) TNF-α concentration, and (D) IL-6 concentration. The alphabet “a” denotes significant difference as compared to hACs Ctr, “b” denotes significant difference as compared to THP-1 Ctr, and “c” denotes significant difference as compared to co-culture Ctr, where p<0.01.

FIG. 6 shows SEM micrographs of (A) hACs cultured in monolayer and (B) co-cultured with activated M1 macrophages after 14 days of treatment under different conditions: (i) no treatment, (ii) micelles encapsulating Dexamethasone (Dex) and (iii) Dex. Scale bars: 10 μm.

### DETAILED DESCRIPTION

The present disclosure relates to an enzymatic and redox responsive polymeric micelle, its method of production as well as its uses. Specifically, use of the enzymatic and redox responsive polymeric micelle for drug delivery.

An aspect of the present disclosure relates to the use of the disclosed polymeric micelles to: a) increase the therapeutic index of a hydrophobic drug through encapsulating the drug in the polymeric micelles; b) reduce systemic side effects through the controlled release profiles of the drug and consequently reducing unnecessary exposure to healthy tissues; and c) increase the therapeutic efficacy of currently used drugs, including anti-inflammatory, anti-cancer and many other therapeutic agents.

In an embodiment, polymeric micelles comprising methoxypolyethylene glycol amine-glutathione-palmitic acid (mPEG-GSH-PA) copolymer were produced. The copolymer was synthetized through 2-step reactions. Firstly, mPEG was covalently linked to GSH using 1-(3-Dimethylaminopropyl)-3-ethylcarbodiimide (EDC) and N-Hydroxysuccinimide (NHS) as coupling agents. Secondly, mPEG-GSH was allowed to react with PA in tetrahydrofuran (THF) with 2-(1H-Benzotriazole-1-yl)-1,1,3,3-tetramethylaminium tetrafluoroborate (TBTU) and triethylamine (TEA) acting as catalysers. After copolymer synthesis, polymeric micelles were prepared through nanoprecipitation.

In an embodiment, after physicochemical characterization of the polymeric micelles, including Fourier-transform infrared spectroscopy (FTIR), particle size, Polydispersity Index (PDI), zeta-potential and morphology analyses, Dex was encapsulated into the polymeric micelles. The Dex content in the polymeric micelles was determined using different micelle:Dex feed weight ratios. The micelle:Dex feed weight ratios varies from 1:0.2 to 1:0.8 for a polymer concentration of 1 mg/mL.

In an embodiment, in vitro drug release profiles under different external stimulations were evaluated using PBS, 10 mM GSH, and 50 mU GR.

In an embodiment, micelles cytocompatibility was assessed in the presence of endothelial cell line (EA.hy926), human monocyte-like cell line (THP-1), and human articular chondrocytes (hACs). A co-culture model of inflammation was also established by culturing hACs and stimulated M1 macrophages.

In an embodiment, the polymeric micelles were characterized.

In an embodiment, tri-block amphiphilic copolymer was synthesized via a two-steps polymerization reaction. Firstly, mPEG reacts with the carboxylic groups of GSH, and then the free amine groups of GSH reacts with the PA. FTIR analysis of the micelles (FIG. 1A) showed a shift of the amine group from the mPEG and GSH (two N—H stretch absorptions at 3300-3000 cm⁻¹) to amide in the polymeric micelles (one N—H stretch absorption at 3300 cm⁻¹ and a C═O peak at 1680-1630 cm⁻¹). Moreover, while the GSH present a weak thiol (S—H) peak at 2550-2620 cm⁻¹, the polymeric micelles presented a weak disulphide (S—S) peak at 700-550 cm⁻¹. The size of the polymeric micelles (FIG. 1B) was 101.3±3.4 nm with a zeta potential of −19.7±3.42 mV and PDI values of 0.092±0.011 (FIG. 1B). Given that the PDI value was lower than 0.2, the micelles population can be considered homogeneous. The assessment of the polymeric micelles' storage stability (FIG. 1C) shows that the micelles are stable over time, without a significant increase in size and PDI for at least 6 months. Based on the SEM analyses (FIG. 1D), the polymeric micelles appear to be largely spherical in shape and has an average diameter of 100 nm. This result is in agreement with the dynamic light scattering (DLS) measurements.

FIG. 1A shows the results of the FTIR analysis of the methoxypolyethylene glycol (mPEG), Glutathione (GSH), Palmitic acid (PA), as well as the polymeric micelles (Mic). FIG. 1B shows the size distribution of the polymeric micelles. FIG. 1C shows the results of the stability evaluation of the polymeric micelles kept in water for 6 months, at 4° C. FIG. 1D shows the SEM micrographs of the polymeric micelles.

In an embodiment, polymeric micelles efficiency as delivery device was assessed using Dex as a model drug. Being a hydrophobic drug, Dex was dissolved in the organic phase of the micelles using different micelle:Dex feed weight ratios. The micelle:Dex feed weight ratios varies from 1:0.2 to 1:0.8 at a polymer concentration of 1 mg/mL (Table 1). The results show that the Dex loading content and entrapment efficiency increases with the feed weight ratio. The maximum entrapment efficiency observed was for micelle:Dex feed weight ratio of 1:0.8 which has an efficiency of about 64%. After entrapment of the drug, the polymeric micelles' size is about 118.8±0.2 nm, with 0.105±0.009 of PDI value and, a zeta potential of −17.4±2.7 mV.

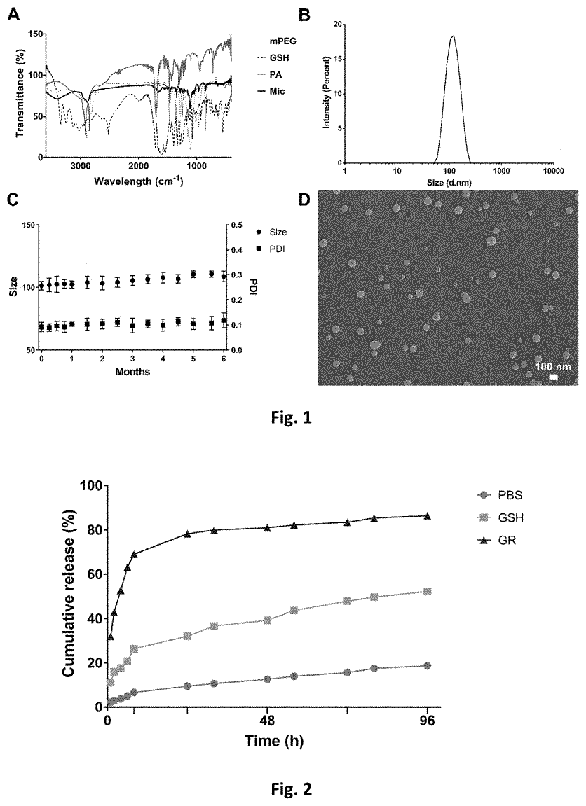

In an embodiment, after quantification of encapsulated Dex into the polymeric micelles, micelle:Dex feed weight ratio of 1:0.8 ratio was chosen for in vitro release profile evaluation (FIG. 2). The in vitro release profile evaluation was performed using the dialysis method in the presence of different artificial milieu. The polymeric micelles showed almost no release during the first 24 hours in the presence of PBS at 37° C. Maximum release of 20% was observed after 5 days. In contrast, the addition of GSH at an intracellular level (10 mM) increased Dex release, maximum release of about 50% was observed after 5 days. The addition of GR at 50 mU was able to induce a burst release of the drug of about 80% during the first 12-24 hours. These results show that the controlled release profiles of the polymeric micelles.

TABLE 1
Dexamethasone (Dex) loading content (mg) and encapsulation
(or entrapment) efficiency (%) into the polymeric
micelles, using different micelle:Dex feed weight
ratios at a polymer concentration of 1 mg/mL.

| Micelles:Dex | Dex loading | Entrapment |
|---|---|---|
| feed weight ratio | content [mg] | efficiency [%] |
| 1:0.2 | 0.57 ± 0.03 | 35.3 ± 1.9 |
| 1:0.4 | 1.38 ± 0.08 | 39.8 ± 1.3 |
| 1:0.6 | 2.50 ± 0.10 | 51.9 ± 2.1 |
| 1:0.8 | 4.65 ± 0.11 | 64.6 ± 1.6 |

FIG. 2 shows the release profile of dexamethasone (Dex) from the polymeric micelles under different artificial milieu: PBS, 10 mM of Glutathione (GSH), and 50 mU of Glutathione Reductase (GR) at 37° C.

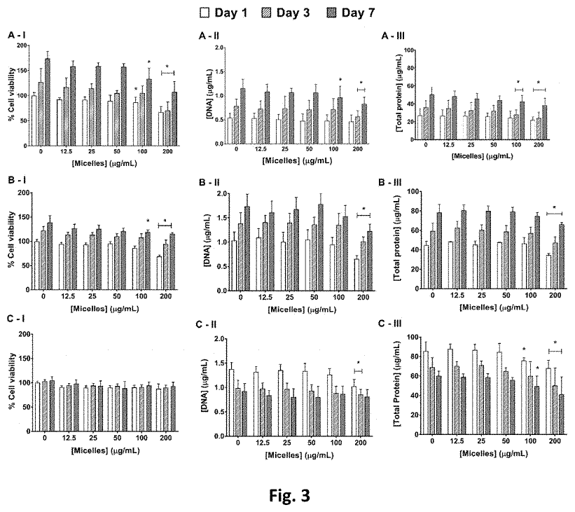

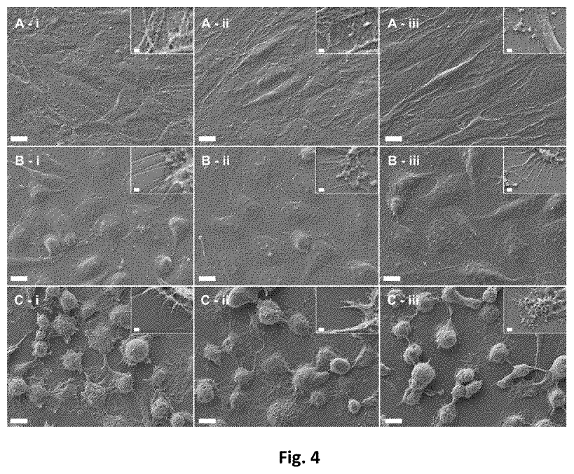

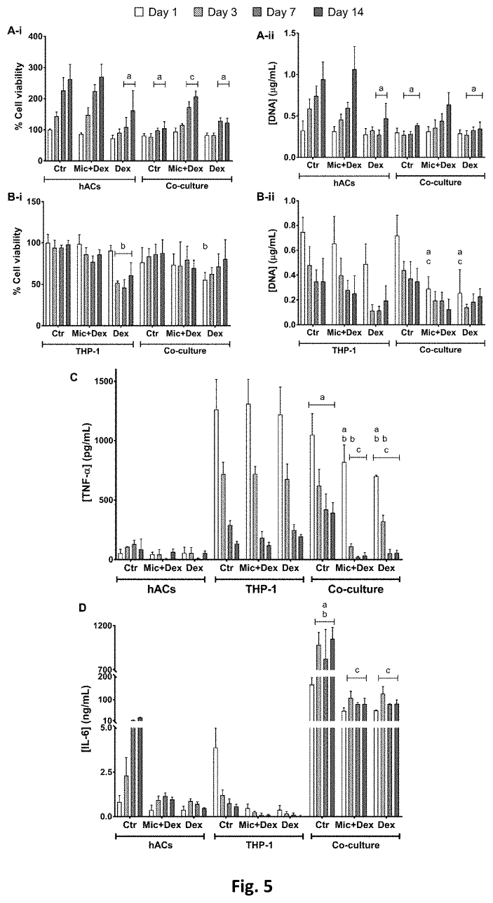

In an embodiment, polymeric micelles' cytocompatibility was analysed. In vitro cellular studies were carried out to assess the viability of different relevant cells that can be affected. The cells used for in vitro cellular analysis are: hACs from diseased knee arthroplasties (phenotype associated with arthritis disease), endothelial cells (main cells of the blood vessels), and macrophages (immune system). After 1, 3 and 7 days of culture, different biological assays were performed to assess cell viability (Alamar Blue—AB—assay), cell proliferation (DNA quantification), total protein synthesis, and cell morphology (SEM). For all the cell types analysed, the results as shown in FIG. 3 revealed that the micelles are cytocompatible until the concentration of the micelle reaches 50 μg/mL. Beyond 50 μg/mL, the polymeric micelles reduced cell viability and cell proliferation as compared with the control. This is especially prominent for polymeric micelles concentration of 200 μg/mL. SEM analyses show that the cell morphology was not affected by the polymeric micelles (FIG. 4). Thus, the preferable concentration of micelles is not more than 50 μg/mL.

FIG. 3 shows the biological performance of (A) hACs, (B) EA cell line, and (C) THP-1 cell line cultured with different concentrations of micelles: (I) cell viability, (II) cell proliferation and (111) total protein synthesis after 1, 3, and 7 days of culture. Asterisk (*) denotes significant differences (p<0.01) as compared to the control (0 μg/mL).

FIG. 4 shows SEM micrographs of the polymeric micelles cultured with (A) hACs, (B) EA cell line and (C) THP-1 cell line in the (I) absence (control) and in the presence of the micelles at (II) 50 μg/mL and (111) 100 μg/mL. Scale bar 10 μm.

In an embodiment, the biological effects of dexamethasone (Dex) in monocultures and co-culture of hACs and THP-1 were analysed. To compare the biological effects of free Dex and the polymeric micelles encapsulating Dex, monoculture and co-culture systems of hACs and stimulated M1 macrophages were used. Three different conditions were tested: (i) no treatment (Ctr), (ii) treatment with micelles encapsulating Dex (Mic+Dex), and (iii) treatment with free Dex (Dex). The concentration of Dex was 100 WI, and in the co-culture system, 50 mU of GR was added.

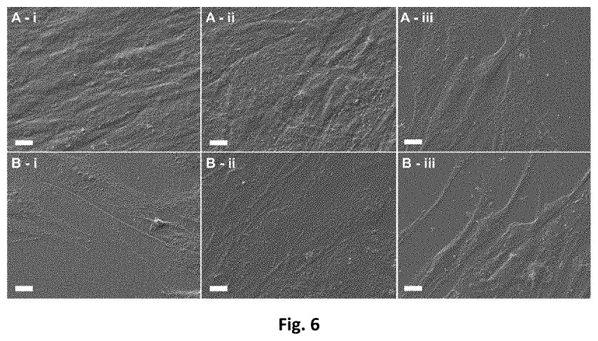

In an embodiment, hACs' viability and proliferation was significantly reduced with the free Dex treatment (FIG. 5A). Interestingly, the encapsulation of Dex into the micelles were able to block Dex's nefarious effects on hACs, as no differences between the Ctr and Mic+Dex groups was observed. In addition, morphological analysis of the hACs (FIG. 6A) shows that the encapsulation of the drug did not affect cell density and morphology as observed in the Dex group. This effect was also observed in the THP-1 cell line (FIG. 5B), where treatment with polymeric micelles showed higher cell viability and proliferation as compared to treatment with free Dex.

In an embodiment, co-culture of hACs with activated M1 macrophages significantly decreased cell viability and proliferation as compared to the hACs control. While treatment with Mic+Dex was able to reduce this harmful effect on chondrocytes, the treatment with free Dex was not. Mic+Dex treatment significantly increases cell viability as compared to co-culture without treatment. These results were also corroborated with morphological analysis of hACs (FIG. 6B). After 14 days of co-culture, hACs showed altered morphology with cell shrinkage and a reduction of cell density. Treatment with polymeric micelles encapsulating Dex was able to prevent cell shrinkage and reduction of cell density to a larger degree as compared to treatment with free Dex. Additionally, the co-culture system not shows any nefarious effects on the THP-1 cells. However, the addition of the Mic+Dex and Dex significantly reduced the quantity of DNA, especially after 1 day of treatment.

In an embodiment, the co-culture of hACs and activated macrophages shows a significant reduction in the amount of pro-inflammatory cytokines produced by those cells. TNF-α and IL-6 cytokines (FIGS. 5C and D, respectively) were quantified in the medium. While the hACs almost do not produce TNF-α, activated M1 macrophages produce around 1.3 ng/mL after 1 day, which was reduced to 0.1 ng/mL after 14 days of treatment. In this case, all conditions (Ctr, Mic+Dex and Dex in THP-1) have a similar reduction of TNF-α in the medium. The polymeric micelles encapsulating Dex were able to reduce more TNF-α in the co-culture system than free Dex, especially after 3 days of treatment. This may be explained due to the controlled release of Dex from the polymeric micelles over the time. With regard to IL-6, the establishment of the co-culture system increased IL-6 amount to a maximum of about 1.2 μg/mL. In this case, both the polymeric micelles with encapsulated Dex and free Dex were able to effectively reduce the amount of IL-6 in the medium to about 0.1 μg/mL.

In an embodiment, the polymeric micelles are able to protect chondrocytes from nefarious effects, such as cell shrinkage and density, of Dex. The encapsulation of Dex in polymeric micelles not compromises the biological action of the drug in inflammation. Additionally, they are able to protect the chondrocytes during inflammation by reducing pro-inflammatory cytokines (TNF-α and IL-6) amount in the media. Therefore, the overall results show that polymeric micelles encapsulating Dex are able to prolong and extend the Dex half-life, as well as increase the efficacy and to reduce some side effects of free Dex.

FIG. 5 shows the biochemical performance of (A) hACs and (B) THP-1 cultured in monolayers and hACs co-cultured with activated M1 macrophages after treatment with different conditions: control (Ctr, no treatment), micelles encapsulating dexamethasone (Mic+Dex) and dexamethasone (Dex). The samples were analyzed for (i) cell viability, (ii) cell proliferation, (C) TNF-α concentration, and (D) IL-6 concentration. The alphabet “a” denotes significant difference compared to the hACs Ctr, “b” denotes significant difference compared to the THP-1 Ctr, and “c” denotes significant difference as compared to the co-culture Ctr where p<0.01.

FIG. 6 shows SEM micrographs of (A) hACs cultured in monolayer and (B) co-cultured with activated M1 macrophages after 14 days of treatment under different conditions: (i) no treatment, (ii) polymeric micelles encapsulating dexamethasone (Dex) and (iii) free Dex. Scale bars: 10 μm.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof.

The above described embodiments are combinable.

### REFERENCES

1. Petros R A, DeSimone J M. Strategies in the design of nanoparticles for therapeutic applications. Nat Rev Drug Discov. 2010; 9(8):615-627.
2. Jhaveri A M, Torchilin V P. Multifunctional polymeric micelles for delivery of drugs and siRNA. Frontiers in pharmacology. 2014; 5:77.
3. Nicolas J, Mura 5, Brambilla D, et al. Design, functionalization strategies and biomedical applications of targeted biodegradable/biocompatible polymer-based nanocarriers for drug delivery. Chem Soc Rev. 2013; 42(3):1147-235.
4. Kamaly N, Yameen B, Wu J, et al. Degradable Controlled-Release Polymers and Polymeric Nanoparticles: Mechanisms of Controlling Drug Release. Chem Rev. 2016; 116(4):2602-63.
5. Pu H L, Chiang W L, Maiti B, et al. Nanoparticles with Dual Responses to Oxidative Stress and Reduced pH for Drug Release and Anti-inflammatory Applications. Acs Nano. 2014; 8(2):1213-1221.
6. Li Y, Xiao K, Luo J, et al. Well-defined, reversible disulfide cross-linked micelles for on-demand paclitaxel delivery. Biomaterials. 2011; 32(27):6633-45.
7. Lu S C. Regulation of glutathione synthesis. Molecular aspects of medicine. 2009; 30(1-2):42-59.
8. Kim H C, Kim E, Ha T L, et al. Highly stable and reduction responsive micelles from a novel polymeric surfactant with a repeating disulfide-based gemini structure for efficient drug delivery. Polymer. 2017433:102-109.
9. Chiang Y T, Yen Y W, Lo C L. Reactive oxygen species and glutathione dual redox-responsive micelles for selective cytotoxicity of cancer. Biomaterials. 2015; 61:150-161.
10. Sredzinska K, Galicka A, Porowska H, et al. Glutathione reductase activity correlates with concentration of extracellular matrix degradation products in synovial fluid from patients with joint diseases. Acta Biochim Pol. 2009; 56(4):635-40.
11. Zhu Z, Du S, Du Y, et al. Glutathione reductase mediates drug resistance in glioblastoma cells by regulating redox homeostasis. Journal of neurochemistry. 2018; 144(1):93-104.

1. A polymeric micelle comprising
a micelle core comprising a palmitic acid that encapsulates a hydrophobic active ingredient; and
a micelle branch comprising glutathione and methoxypolyethylene glycol, wherein the methoxypolyethylene glycol is bonded to glutathione
the micelle core and micelle branch forming a methoxypolyethylene glycol amine-glutathione-palmitic acid;
wherein the methoxypolyethylene glycol amine-glutathione-palmitic acid forms a copolymer with a hydrophobic active ingredient encapsulated.

2. (canceled)

3. The polymeric micelle of claim 1 wherein the polymeric micelle is enzymatic and redox responsive.

4. A pharmaceutical composition comprising an effective amount of the polymeric micelle of claim 1.

5. The polymeric micelle of claim 1 wherein the polymeric micelle is a vehicle for drug delivery.

6. A method of treating inflammatory diseases in a subject, the method comprising administering the polymeric micelle of claim 1 to the subject.

7. A method of treating osteoarthritis or rheumatoid arthritis in a subject, the method comprising administering the polymeric micelle of claim 1 to the subject.

8. The polymeric micelle of claim 1 wherein the active ingredient is a hydrophobic drug.

9. The polymeric of claim 1 wherein the hydrophobic drug is dexamethasone, prednisolone, betamethasone, or combinations thereof.

10. The polymeric micelle of claim 1 wherein the size of the micelle is at least 100 nm.

11. The polymeric micelle of claim 1 wherein the micelle has an encapsulation efficiency from 30% to 70%.

12. The polymeric micelle of claim 1 wherein the micelle to drug ratio feed weight is in the range of from 1:0.2 to 1:0.8 for a polymer concentration of 1 mg/mL.

13. The polymeric micelle of claim 1 wherein the amount of hydrophobic active ingredient is from 0.2 mg to 6.0 mg.

14. The polymeric micelle of claim 1 wherein the hydrophobic active ingredient is dexamethasone and wherein the polymeric micelle releases the dexamethasone when the glutathione reductase concentration is at least 50 mU.

15. The polymeric micelle of claim 1 wherein the hydrophobic active ingredient is dexamethasone and wherein the polymeric micelle releases the dexamethasone when the glutathione concentration is at least 10 μM.

16. A pharmaceutical composition comprising the polymeric micelles of claim 1 and a suitable pharmaceutical vehicle.

17. The pharmaceutical composition of claim 16 wherein the concentration of the polymeric micelles is less than or equal to 50 μg/mL.

18. The pharmaceutical composition of claim 16 wherein the composition is a suspension for systemic administration.

19. A method of producing the polymeric micelles of claim 1 comprising:
covalently linking methoxypolyethylene glycol amine and glutathione using coupling agents to form a first copolymer;
adding the first copolymer to palmitic acid in tetrahydrofuran to form a second copolymer;
nano-precipitating the second copolymer to form the polymeric micelles.

20. The method of claim 19 wherein the coupling agents are 1-(3-Dimethylaminopropyl)-3-ethylcarbodiimide and N-Hydroxysuccinimide.

21. The method of claim 19 further comprising:
adding 2-(1H-Benzotriazole-1-yl)-1,1,3,3-tetramethylaminium tetrafluoroborate (TBTU) and triethylamine (TEA) as catalysers when forming the second copolymer.
